(19) United States
(12) Patent Application Publication
(10) Pub. No.: US 2005/0209618 A1
(43) Pub. Date: Sep. 22, 2005

(54) Rigid shafted instrumentation for vitreoretinal surgery

(76) Inventor: Michael D. Auld, Chesterfield, MO (US)

Correspondence Address: Kevin L. Klug ( Attorney), 11237 Concord Village Ave., St. Louis, MO, 63123-6903, US

(21) Appl. No.: 11/070,788

(22) Filed: Mar. 2, 2005

Related U.S. Application Data
(60) Provisional application No. 60/550,357, filed on Mar. 5, 2004.

(51) Int. Cl. A61F 9/00

(52) U.S. Cl. 606/166

(57) ABSTRACT

A rigid shafted instrumentation for vitreoretinal surgery device utilizing a 20 gauge or smaller shaft manufactured of tungsten carbide or equivalent material. The material of the present invention allows utilization of very small diameter shafts which provide less trauma to the eye without the bending of prior art materials. The rigid shaft material is utilized with a plurality of vitreoretinal surgical devices such as ophthalmic picks, probes, knives, light pipes, scissors, or forceps.

This application claims priority of Provisional Patent Application No. 60/550,357, filed Mar. 5, 2004.

### BACKGROUND OF THE INVENTION

The art of the present invention relates to shafting for vitreoretinal or eye surgical instruments in general and more particularly to an apparatus and method which utilizes smaller diameter (i.e. larger gauge) shafts of tungsten carbide which provide less shaft bending or deflection while utilizing a smaller diameter shaft for the aforesaid eye surgical instruments.

Current trends in posterior segment ophthalmic surgery are driving the development of ever smaller single shafted instrumentation. The introduction and mainstream adoption of sub 20 gauge surgery has been heretofore limited by inadequate instrument shaft stiffness.

As instrument gauge increases (i.e. diameter decreases), the resultant stiffness of the instrument decreases proportionally. As the instrument is manipulated by the surgeon, the instrument shaft is compelled to resist the bending forces applied by the tissues surrounding the surgical sight. In vitreous surgery, the instrument shaft is in continuous contact with the eyewall entry sight (typically pars plana), which applies a force equal and opposite to that of the surgeons intended manipulations. As instrument size approaches 25 gauge, the flexibility of the instrument typically limits the surgeons ability to finely manipulate the instrument tip, as the tip will move in unintended directions and amounts as the shaft is bent by the eyewall torque.

Conventional surgical instrument construction typically utilizes conventional stainless steel alloys and stainless hypodermic tubing for the instrument shaft. These materials can be cold worked and tempered to achieve high strength values, but the strength values are not the primary contributor to shaft stiffness (deflection) characteristics. It is the elastic modulus (Youngs Modulus) value which determines the deflection of a given engineering alloy. Typical values for surgical stainless alloys modulus is approximately 28 KSI, and remains invariant with heat treatment.

The present invention apparatus and method utilizes the use of specialty alloys for the instrument shaft which will impart a significant improvement in shaft deflection characteristics. Particularly, tungsten carbide alloys can be utilized which typically have a 3 to 4 times higher modulus of elasticity than the ferrous stainless steels. These alloys will allow the surgeon to manipulate the instrument with limited regard to untended tip vectors caused by shaft bending. Instrument types including single shafted manipulators, picks, endo probes, light pipes, surgical knives, as well as vitreoretinal scissors and forceps can benefit from this improved shaft deflection characteristic.

Accordingly, it is an object of the present invention to provide a rigid shafted instrumentation for vitreoretinal surgery having a shaft of a rigid material such as tungsten carbide which does not flex or bend as does the prior art.

### SUMMARY OF THE INVENTION

To accomplish the foregoing and other objects of this invention there is provided a rigid shafted instrumentation for vitreoretinal surgery device.

The present art device therefore incorporates and utilizes in a preferred embodiment, a tungsten carbide shaft having one or more of the aforesaid picks, probes, or knives attached at an first end or which have integral light pipes, scissors, or forceps attached or formed therewith in order to provide the aforesaid stiffness desired. A second end of said shaft contains a handle form for manipulation or activation of said surgical tools. Alternative embodiments utilize other alloys which typically have a 3 to 4 times higher modulus of elasticity than the ferrous stainless steels in the aforesaid applications. Further alternative embodiments utilize a two or more part shaft whereby a portion nearest said first end (distal portion) utilizes a more limp or flexible material and a proximal portion nearest said handle utilizes the stiffer shafting described herein.

### BRIEF DESCRIPTION OF THE DRAWINGS

Numerous other objects, features and advantages of the invention should now become apparent upon a reading of the following detailed description taken in conjunction with the accompanying drawings, in which:

FIG. 1 is a plan view of the preferred embodiment of the rigid shafted instrumentation for vitreoretinal surgery showing a typical utilization within a cross section of an eye globe.

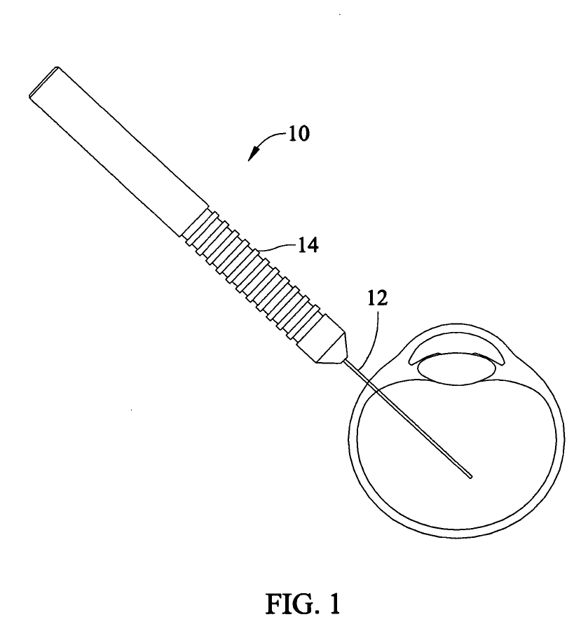

FIG. 2 is a plan view of the prior art in comparison with the present art shown as a phantom shaft showing a typical utilization within a cross section of an eye globe.

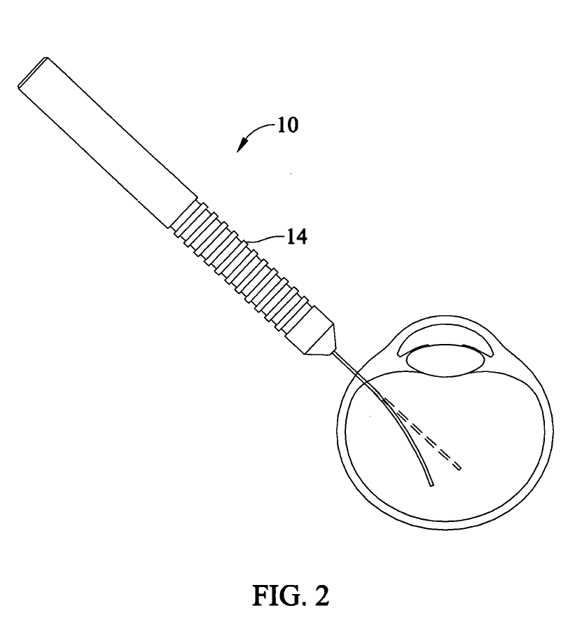

FIG. 3 is a plan view of an alternate embodiment or configuration whereby said shaft is comprised of a two part proximal stiff and a distal non-stiff or limp material.

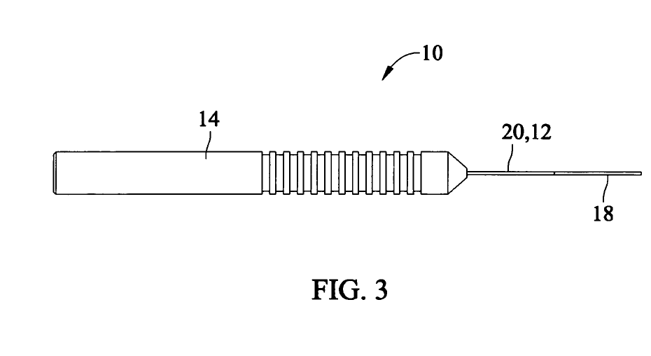

FIG. 4 is a plan cross sectional view of an alternate embodiment showing the flexible tubing around said shaft.

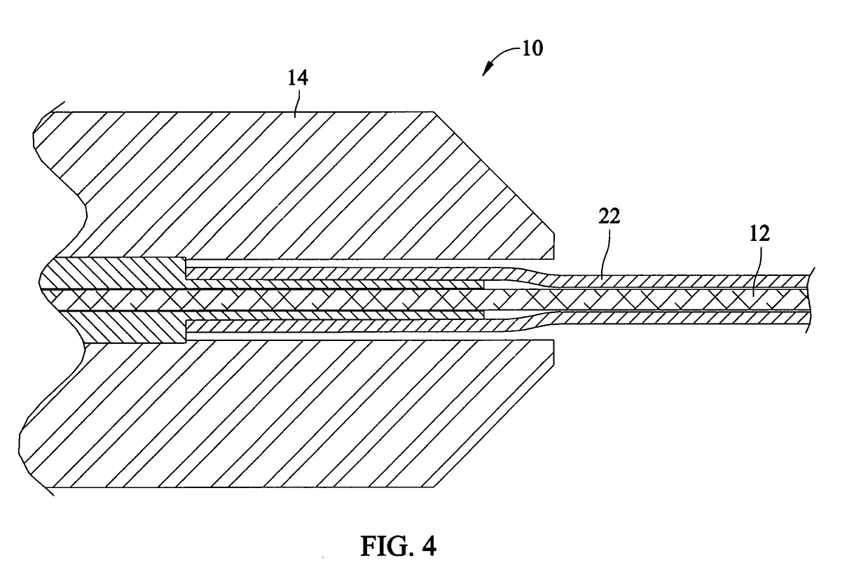

FIG. 5 is a perspective view of the present art compared with the prior art eye surgical tool showing the stiffness comparison by pushing one shaft against the other and observing the flex in the prior art shaft. This Figure utilizes a photograph as the only practicable medium for illustrating the claimed invention as a whole pursuant to 37 CFR 1.84.

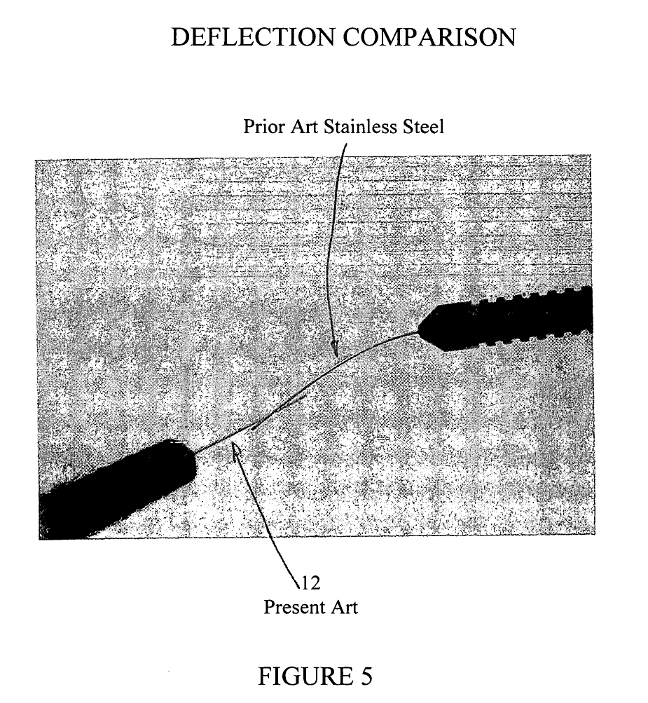

### DETAILED DESCRIPTION

Referring now to the drawings, there is shown in the Figures a preferred embodiment of a rigid shafted instrumentation for vitreoretinal surgery 10.

The present art device incorporates and utilizes in a preferred embodiment, a tungsten carbide shaft 12 having one or more of the aforesaid picks, probes, or knives attached at an first end or which have integral light pipes, scissors, or forceps attached or formed therewith in order to provide the aforesaid stiffness desired. (tungsten carbide is metallurgically represented as “WC”) A second end of said shaft contains a handle 14 form for manipulation or activation of said surgical tools. Alternative embodiments utilize other alloys which typically have a 3 to 4 times higher modulus of elasticity than the ferrous stainless steels in the aforesaid applications. Further alternative embodiments utilize a two or more part shaft 16 whereby a portion nearest said first end (distal portion) 18 utilizes a more limp or flexible material and a proximal portion 20 nearest said handle 14 utilizes the stiffer shafting described herein, i.e. tungsten carbide or equivalent. For a light pipe configuration, said shaft has a core fiber optic within said shaft 12. That is, the shaft 12, preferably of tungsten carbide, is hollow with an optical fiber within the hollow portion.

Further alternative embodiments utilize a super thin, typically 0.0005 inch to 0.001 inch thickness, flexible tubing 22 around the shaft 12 to contain the shaft 12 should it shatter or break. That is, since tungsten carbide is typically a more brittle material, it could leave remnants in the eye should it break. By surrounding the shaft with a flexible tubing 22, should breakage occur, remnants will remain within said tubing 22. Typically said tubing 22 is a mylar heat shrink tubing available from a plurality of companies including Advanced Polymers, Inc. of Salem, N.H. Said tungsten carbide shaft material is also available from a plurality of companies including Extramet Products, LLC, of Latrobe, Pa.

Those skilled in the art will appreciate that a rigid shafted instrumentation for vitreoretinal surgery apparatus and method has been presented to maximize shaft stiffness for surgical instruments which heretofore has not been utilized. The apparatus and method of practice allows for surgical instrument manipulation with a small diameter shaft with a minimum of shaft deflection, especially for eye surgical instruments having 20 gauge or smaller shafts.

Having described the invention in detail, those skilled in the art will appreciate that modifications may be made of the invention without departing from its spirit. Therefore, it is not intended that the scope of the invention be limited to the specific embodiments illustrated and described. Rather it is intended that the scope of this invention be determined by the appended claims and their equivalents.

1. A rigid shafted instrumentation for vitreoretinal surgery comprising:
single shafted ophthalmic surgery instrument having a handle and a shaft of 20 gauge or smaller; and
said shaft comprising a tungsten carbide material whereby said shaft retains stiffness.

2. The rigid shafted instrumentation for vitreoretinal surgery as set forth in claim 1 further comprising:
a flexible tubing surrounding said shaft, whereby said shaft is contained should breakage occur.

3. The rigid shafted instrumentation for vitreoretinal surgery as set forth in claim 2, said flexible tubing further comprising:
a mylar heat shrink tubing.

4. The rigid shafted instrumentation for vitreoretinal surgery as set forth in claim 1 said single shafted ophthalmic surgery instrument comprising:
a light pipe having a core optical fiber within said shaft, said shaft having a hollow portion and said optical fiber said hollow portion.

5. The rigid shafted instrumentation for vitreoretinal surgery as set forth in claim 1 said single shafted ophthalmic surgery instrument comprising:
an ophthalmic probe.

6. The rigid shafted instrumentation for vitreoretinal surgery as set forth in claim 1 said single shafted ophthalmic surgery instrument comprising:
an ophthalmic pick.

7. The rigid shafted instrumentation for vitreoretinal surgery as set forth in claim 1 said single shafted ophthalmic surgery instrument comprising:
an ophthalmic knife.

8. A rigid shafted instrumentation for vitreoretinal surgery comprising:
single shafted ophthalmic surgery instrument having a handle and a shaft of 20 gauge or smaller; and
said shaft having two parts, said two parts comprising a first distal end portion and a second proximal end portion; and
said proximal end portion of said shaft comprising a tungsten carbide material whereby said shaft retains stiffness.

9. The rigid shafted instrumentation for vitreoretinal surgery as set forth in claim 8 further comprising:
a flexible tubing surrounding said shaft, whereby said shaft is contained should breakage occur.

10. A method for performing vitreoretinal surgery, the steps comprising:
forming a single shaft of an ophthalmic surgery instrument from tungsten carbide of 20 gauge or less; and
forming a handle for said shaft; and
placing said handle on said shaft; and
inserting said shaft into a ball of an eye; and
performing a surgical procedure with said shaft; and
moving said shaft with minimal bending of said shaft.
